(19) United States
(12) Patent Application Publication
(10) Pub. No.: US 2011/0190831 A1
(43) Pub. Date: Aug. 4, 2011

(54) STEERABLE BALLOON CATHETER

(75) Inventors: Masoumeh Mafi, Mountain View, CA (US); Johanah M. Soliman, San Jose, CA (US); Warren C. Sapida, Santa Clara, CA (US); Samuel V. Bolosan, San Jose, CA (US)

(73) Assignee: KYPHON SARL, Neuchatel (CH)

(21) Appl. No.: 12/696,848

(22) Filed: Jan. 29, 2010

(51) Int. Cl. A61B 17/56; A61M 25/10

(52) U.S. Cl. 606/86 R; 604/96.01

(57) ABSTRACT

A balloon catheter can include an actively steerable element for adjusting the configuration of the balloon after placement at a target location. By coupling the actively steerable element to the balloon, any articulation of the actively steerable element will also re-configure the position of the balloon, thereby enabling greater control over procedures that make use of such a balloon catheter. The active steering capability can also enhance the material manipulation capabilities of the balloon catheter, and enable operations and actions that are not possible with a non-steerable balloon catheter.

### FIELD OF THE INVENTION

The invention relates to a system and method for performing a surgical procedure, and in particular, to an articulating balloon catheter.

### BACKGROUND OF THE INVENTION

A minimally invasive procedure is a medical procedure that is performed through the skin or an anatomical opening. In contrast to an open procedure for the same purpose, a minimally invasive procedure will generally be less traumatic to the patient and result in a reduced recovery period.

However, there are numerous challenges that minimally invasive procedures present. For example, minimally invasive procedures are typically more time-consuming than their open procedure analogues due to the challenges of working within a constrained operative pathway. In addition, without direct visual feedback into the operative location, accurately selecting, sizing, placing, and/or applying minimally invasive surgical instruments and/or treatment materials/devices can be difficult.

For example, for many individuals in our aging world population, undiagnosed and/or untreatable bone strength losses have weakened these individuals' bones to a point that even normal daily activities pose a significant threat of fracture. In one common scenario, when the bones of the spine are sufficiently weakened, the compressive forces in the spine can cause fracture and/or deformation of the vertebral bodies. For sufficiently weakened bone, even normal daily activities like walking down steps or carrying groceries can cause a collapse of one or more spinal bones. A fracture of the vertebral body in this manner is typically referred to as a vertebral compression fracture. Other commonly occurring fractures resulting from weakened bones can include hip, wrist, knee and ankle fractures, to name a few.

Fractures such as vertebral compression fractures often result in episodes of pain that are chronic and intense. Aside from the pain caused by the fracture itself, the involvement of the spinal column can result in pinched and/or damaged nerves, causing paralysis, loss of function, and intense pain which radiates throughout the patient's body. Even where nerves are not affected, however, the intense pain associated with all types of fractures is debilitating, resulting in a great deal of stress, impaired mobility and other long-term consequences. For example, progressive spinal fractures can, over time, cause serious deformation of the spine (“kyphosis”), giving an individual a hunched-back appearance, and can also result in significantly reduced lung capacity and increased mortality.

Because patients with these problems are typically older, and often suffer from various other significant health complications, many of these individuals are unable to tolerate invasive surgery. Therefore, in an effort to more effectively and directly treat vertebral compression fractures, minimally invasive techniques such as vertebroplasty and, subsequently, kyphoplasty, have been developed. Vertebroplasty involves the injection of a flowable reinforcing material, usually polymethylmethacrylate (PMMA—commonly known as bone cement), into a fractured, weakened, or diseased vertebral body. Shortly after injection, the liquid filling material hardens or polymerizes, desirably supporting the vertebral body internally, alleviating pain and preventing further collapse of the injected vertebral body.

Because the liquid bone cement naturally follows the path of least resistance within bone, and because the small-diameter needles used to deliver bone cement in vertebroplasty procedure require either high delivery pressures and/or less viscous bone cements, ensuring that the bone cement remains within the already compromised vertebral body is a significant concern in vertebroplasty procedures. Kyphoplasty addresses this issue by first creating a cavity within the vertebral body (e.g., with an inflatable balloon) and then filling that cavity with bone filler material. The cavity provides a natural containment region that minimizes the risk of bone filler material escape from the vertebral body. An additional benefit of kyphoplasty is that the creation of the cavity can also restore the original height of the vertebral body, further enhancing the benefit of the procedure.

Conventional kyphoplasty systems use balloon catheters that can be inflated to a desired size by the physician. Inflation is performed once the balloon catheters are placed within the bone (typically using a transpedicular approach). Therefore, the final positioning and configuration of the actual balloons is defined solely by the placement of the balloon catheter. However, in some instances, the as-placed position of the balloon may not be optimal for the procedure (e.g., configuring the balloon such that inflation occurs towards the anterior of the vertebral body can enhance the mechanical advantage provided by the balloon during inflation). Unfortunately, conventional balloon catheters do not allow such “post-placement” repositioning of the balloon.

Accordingly, it is desirable to provide surgical tools and techniques that enable adjustment of placement in-situ.

### SUMMARY OF THE INVENTION

By incorporating an actively steerable element into a balloon catheter, repositioning of the balloon can be beneficially performed after the balloon catheter has been inserted into the target surgical location.

In one embodiment, a balloon catheter can include an elongate shaft coupled to a balloon, a steering mechanism extending along the shaft and positioning a steerable element into or adjacent the balloon, and an actuator for articulating the steerable element. In various embodiments, the steering mechanism can be positioned within the elongate shaft, and can optionally be placed within an inner catheter within the elongate shaft. In various other embodiments, the steering mechanism can be positioned adjacent to the elongate shaft.

Manipulation of the actuator articulates the steerable element such that the configuration of the balloon is changes. In doing so, the positioning/placement of the balloon during a surgical procedure can be adjusted as desired by the physician to achieve a desired outcome.

In various embodiments, an actively steerable balloon catheter can be used in a kyphoplasty procedure to allow adjustment the positioning and/or placement of the balloon within the vertebral body. In so doing, the procedure can be performed using a unilateral approach while still providing proper bone filler material placement for good structural support. However, in other embodiments, the actively steerable balloon catheter can be used in conventional bilateral procedures, or other surgical procedures.

As will be realized by those of skilled in the art, many different embodiments of an balloon catheter incorporating active steering capabilities, along with systems, kits, and/or methods of using such a balloon catheter according to the present invention are possible. Additional uses, advantages, and features of the invention are set forth in the illustrative embodiments discussed in the detailed description herein and will become more apparent to those skilled in the art upon examination of the following.

### BRIEF DESCRIPTION OF THE DRAWINGS

FIGS. 1A-1B show an exemplary balloon catheter that incorporates a steering element for in-situ balloon positioning.

FIGS. 2A-2B show an exemplary steering element for use in a balloon catheter.

FIG. 3 shows a kit that includes a balloon catheter that incorporates a steering element for in-situ balloon positioning.

FIGS. 4A-4F show an exemplary kyphoplasty procedure using an expandable bone tamp incorporating a steering element for in-situ balloon positioning.

FIG. 5 shows a flow diagram for performing a surgical procedure using an in-situ steerable balloon catheter.

### DETAILED DESCRIPTION

By incorporating an actively steerable element into a balloon catheter, repositioning of the balloon can be beneficially performed after the balloon catheter has been inserted into the target surgical location.

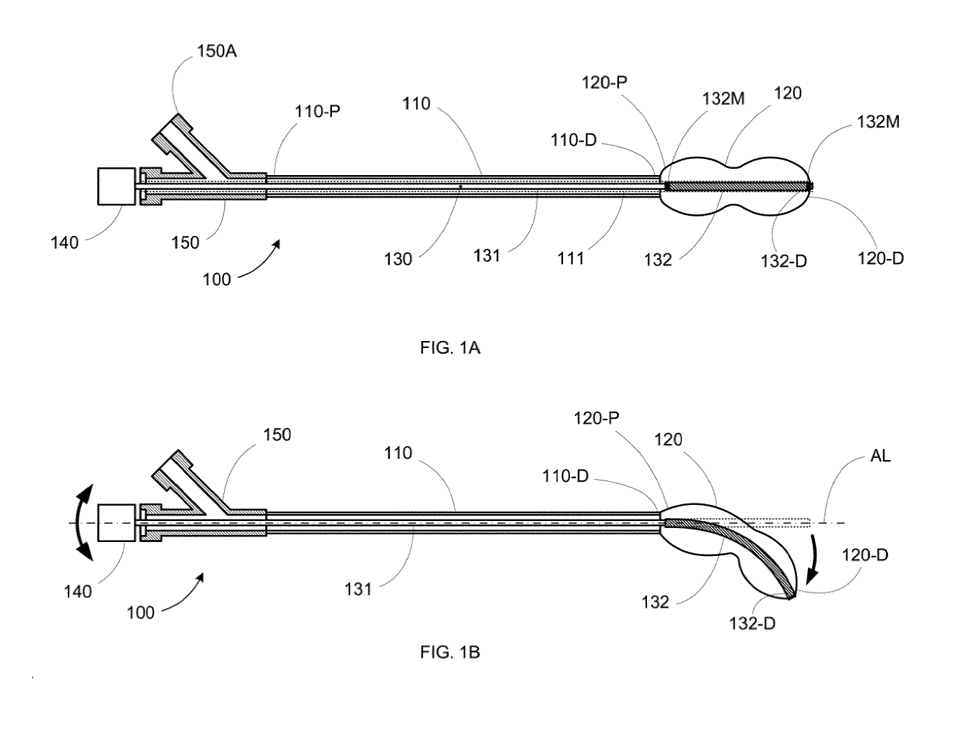

FIG. 1A shows a cross-section of an embodiment of a balloon catheter 100 that can be used in a surgical procedure, such as balloon kyphoplasty. Balloon catheter 100 includes a shaft 110, an inflatable structure (e.g., balloon) 120, a steering mechanism 130, an actuator 140 for controlling steering mechanism 130, and a connector 150. Inflatable structure 120 can be formed from a compliant (e.g., latex), semi-compliant (e.g., polyurethane), or non-compliant (e.g., nylon) material. Although depicted as a single-chamber “peanut”-shaped balloon for exemplary purposes, balloon 120 can have any shape and/or construction (e.g., a spherical balloon, a multi-chamber balloon, or a balloon with internal or external shaping/reinforcing features, among others).

Inflatable structure 120 is coupled to a distal end 110-D of shaft 110, and connector 150 is coupled to a proximal end 110-P of shaft 110. Connector 150 includes a port 150A (e.g., a Luer lock connection) for receiving inflation material (e.g., saline solution or contrast solution) for inflating balloon 120. Note that in various embodiments, connector 150 can include any number of any type of ports.

Steering mechanism 130 includes a steerable element 132 and a shaft 131 that couples steerable element 132 to actuator 140. Steerable element 132 can be configured into a variety of shapes (i.e., articulated) by actuator 140 without any external restraint, and is therefore “actively” steerable (in contrast to a passive structure like a bent shape-memory wire that can only be straightened by being placed in an external sleeve or sheath).

Note also that minimally invasive procedures such as kyphoplasty are typically performed under fluoroscopy, so that the physician can at least have some visual indication of the surgical activity within the patient. Therefore, in some embodiments, optional radiopaque markers 132M can be placed at various locations on steerable element 132 to facilitate positioning of balloon catheter 100 in the patient. In various other embodiments, steerable element 132 can be formed from, or can include, radiopaque material(s).

Because steerable element 132 extends into balloon 120 such that reconfiguration of steerable element 132 by actuator 140 (e.g., as shown in FIG. 1B) also changes the shape of balloon 120. In various embodiments, steerable element can be coupled to a distal end 120-D of balloon 120 (or any other location on the inside or outside of balloon 120). In various other embodiments, balloon catheter 100 can include an optional inner catheter 111 within shaft 110, with the distal end of inner catheter 111 being coupled to the distal end 120-D of balloon 120. Steerable element 132 could then be positioned within, or outside of, inner catheter 111 within balloon 120 to provide steering control over balloon 120. In various other embodiments, inner catheter 111 could accept a stiffening stylet or guidewire (not shown for simplicity), with steering mechanism 130 either sharing the space within inner catheter 111 or being positioned outside of inner catheter 111. Finally, in various other embodiments, steerable element 132 can simply extend into balloon 120 with its distal end 132-D completely unattached.

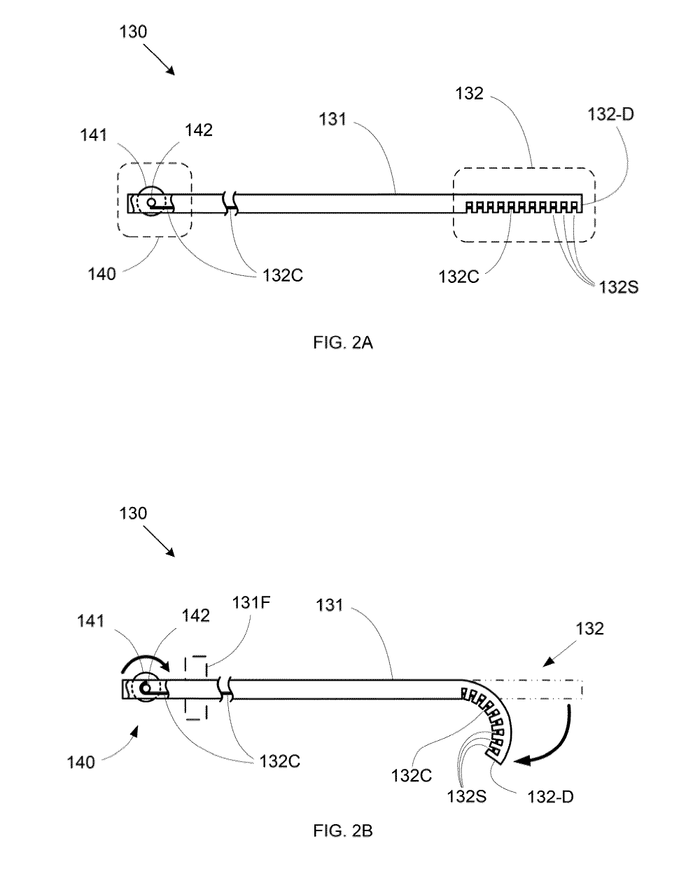

FIGS. 2A and 2B show an exemplary embodiment of steering mechanism 130, in which steerable element 132 includes a series of slots 132S formed in shaft 131. A cable 132C is attached to the distal end 132-D of steerable element 132 and runs slidably through shaft 131 to actuator 140. Actuator 140 includes a spindle 142 mounted on a thumbwheel 141, with cable 132C attached to spindle 142.

Rotating thumbwheel 141 as shown in FIG. 2B winds cable 132C around spindle 142, thereby causing slotted steerable element 132 to curl away from the longitudinal axis of shaft 131. Slots 132S determine the direction of curvature for steerable element 132. In one embodiment, shaft 131 includes features 131F (e.g., flanges, a collar, ribs, or extensions, among others) that facilitate rotation of steering mechanism (in various other embodiments, such features can be placed elsewhere on balloon catheter 100).

In various embodiments, shaft 131 can be formed from shape-memory material (e.g., Nitinol) so that once cable 132C is allowed to unspool from spindle 142 (e.g., by releasing or unlocking thumbwheel 141), steerable element 132 returns to its original (straight) configuration. In various other embodiments, cable 132C can be selected to have sufficient axial rigidity to “push” steerable element 132 back into a straight configuration. In various other embodiments, steering mechanism 130 can include multiple cables to control the configuration of steerable element 132. For example, in one embodiment, steering mechanism 130 can include a second cable in opposition to cable 132S to flex steerable element 132 back to a straight condition (or even to curve in a different direction).

Note that while steering mechanism 130 is depicted as having steerable element 132 formed as a slotted shaft for exemplary purposes, steerable element 132 can have any construction that provides active steering capability at steerable element 132. For example, in various embodiments, steerable element 132 could include a flexible sleeve over a flexible internal member between parallel control cables, such that each cable pulls the flexible member in a different direction. In various other embodiments, steerable element 132 could include a coil of wire surrounding a relatively rigid core that pushes distally to flex the coil. Various other embodiments will be readily apparent.

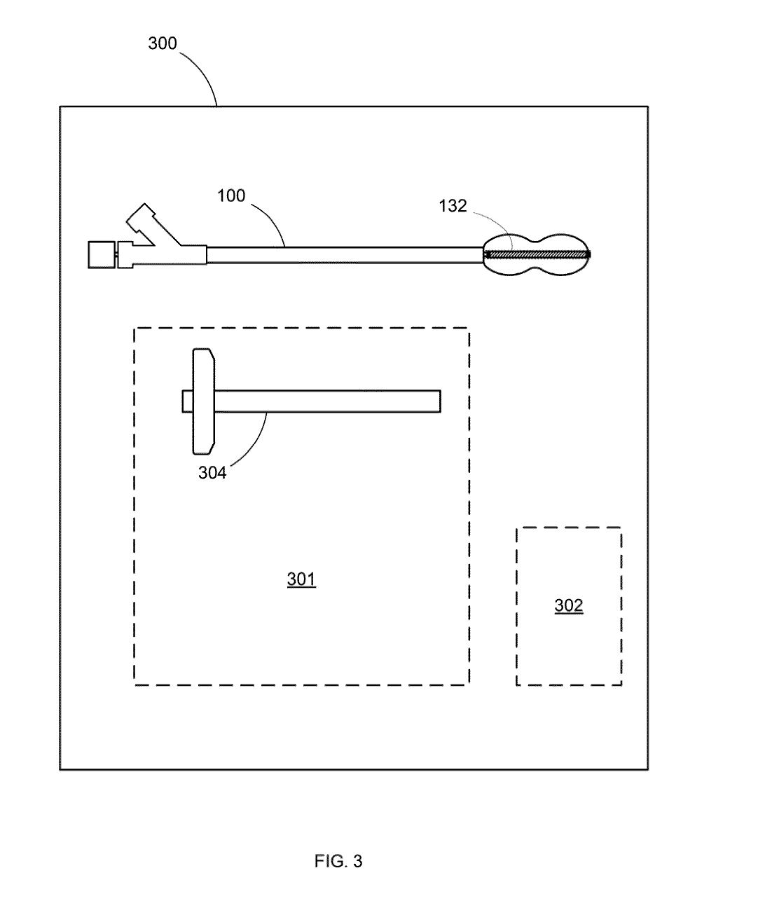

FIG. 3 shows a diagram of a kit 300 for use in performing a surgical procedure (e.g., balloon kyphoplasty) as described in greater detail below. Kit 300 includes a balloon catheter 100 that includes an actively steerable element 132 (e.g., as described above with respect to FIGS. 1A-1B, 2A-2B). In various embodiments, kit 300 can further include optional additional instruments 301, such as a cannula 304 sized to receive balloon catheter 100, an introducer, guide pin, drill, curette, and/or access needle, among others (only cannula 404 is shown for simplicity). In various other embodiments, kit 300 can further include optional directions for use 302 that provide instructions for using balloon catheter 100 and optional additional instruments 301 (e.g., instructions for performing a balloon kyphoplasty procedure using balloon catheter 100 and optional additional instruments 301).

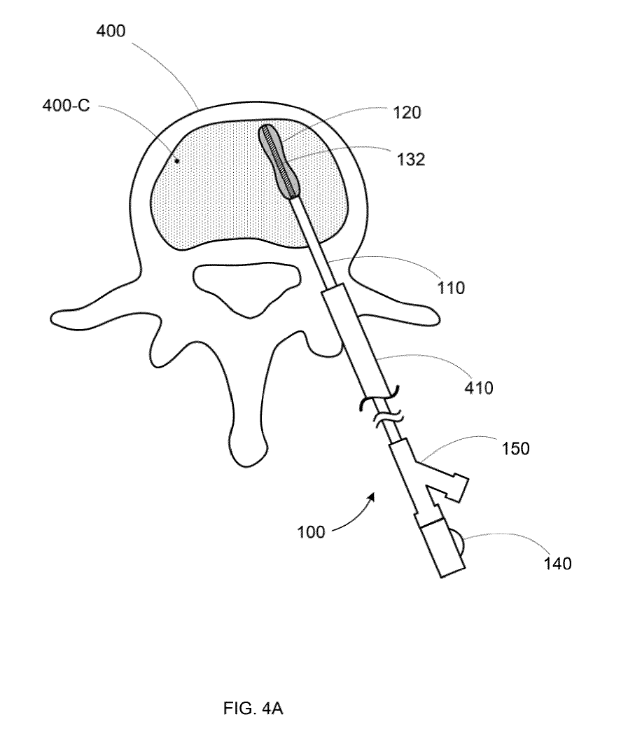

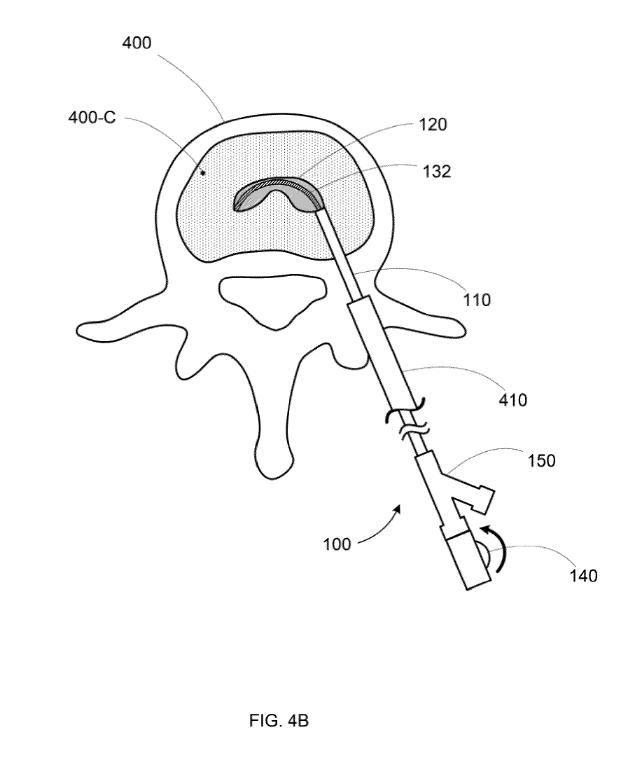

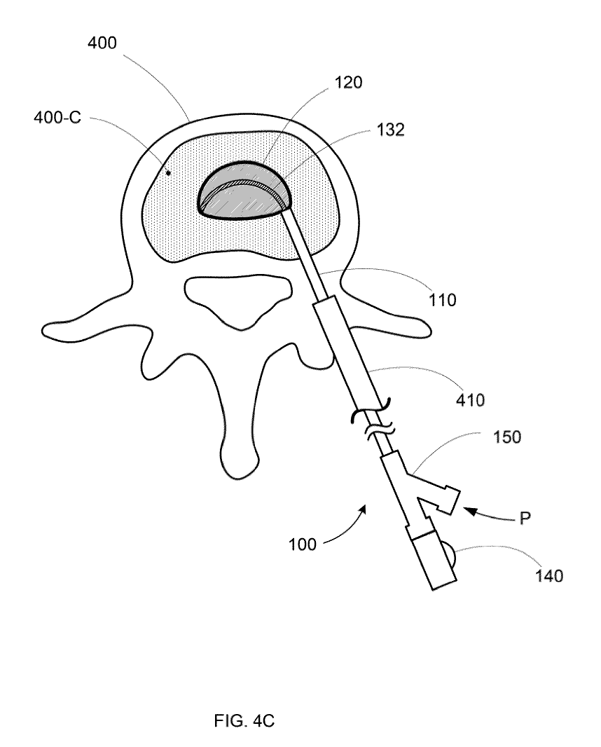

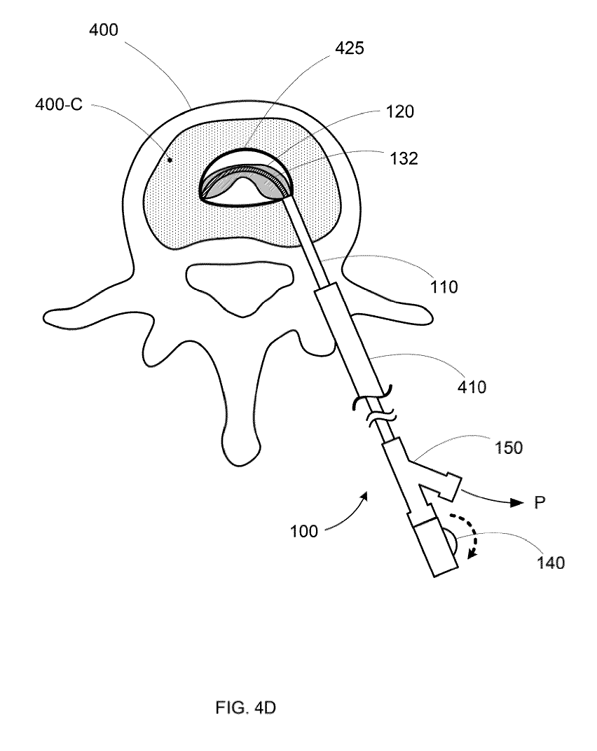

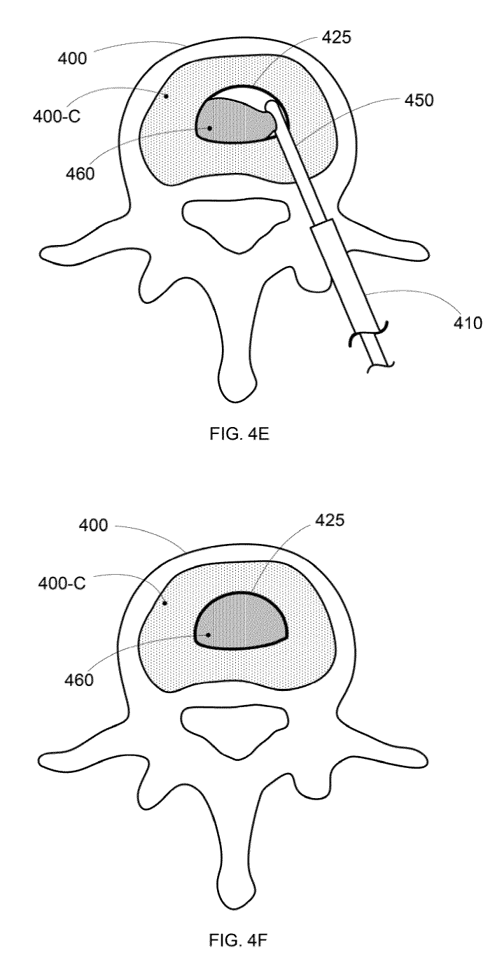

FIGS. 4A-4F show an exemplary kyphoplasty procedure using a balloon catheter 100 that incorporates an actively steerable element 132 (as described with respect to FIGS. 1A-1B). Note that while a unilateral procedure (i.e., use of a single balloon catheter) is depicted for exemplary purposes, in various other embodiments any number of balloon catheters 100 can be used. In some embodiments, actively steerable balloon catheter 100 can be used with conventional (i.e., not actively steerable) balloon catheters.

FIG. 4A shows cross-sectional transverse view of a portion of a human vertebral column having a vertebra 400. Vertebra 400 has collapsed due to a vertebral compression fracture (VCF) that could be the result of osteoporosis, cancer-related weakening of the bone, and/or physical trauma. The resulting abnormal curvature of the spine caused by such a fracture can lead to severe pain and further fracturing of adjacent vertebral bodies.

In FIG. 4A, a cannula 410 is positioned within fractured vertebra 400, thereby providing an access path to the target surgical location, which in this case is the cancellous bone structure 400-C within vertebra 400. Typically, cannula 410 would be docked into the exterior wall of vertebral body 400 (via either a transpedicular or extrapedicular approach) using a guide needle and/or dissector, after which a drill or other access tool (not shown) could be used to create a path further into cancellous bone 400-C. However, any other method of cannula placement can be used. Balloon catheter 100 is inserted into cannula 410 to position balloon 120 within cancellous bone 400-C.

Then, as shown in FIG. 4B, actuator 140 is used to change the configuration of steerable element 132, in this example causing steerable element 132 to curve inward and away from the exterior wall of vertebral body 400. Consequently, balloon 120 is similarly repositioned by steerable element 132.

In some embodiments, a curette or other mechanical tool can be used to break up or scrape away a portion of cancellous bone 400-C prior to the insertion of balloon catheter 100 into vertebral body 400. In this manner, the resistance encountered by steerable element 132 as it moves within vertebral body 400 can be minimized.

However, besides providing greater positional control over balloon 120, the active steering functionality of steerable element 132 can also provide force generation capabilities that are significantly greater than would be possible from passive shaping elements (e.g., a wire with a preformed bend positioned within balloon 120). Therefore, in various embodiments, balloon catheter 100 itself can be used to scrape, cut, and/or compact cancellous bone 400-C through the articulation of steerable element 132.

Note that while the placement and positioning of balloon 120 is described as a sequential two-step process (i.e., insert balloon catheter 100 into vertebra 300 and then articulate steerable element 132) for exemplary purposes, any number and sequence of placement and positioning steps can be performed. For example, in one embodiment, balloon 120 could be placed in cancellous bone, steerable element 132 could be articulated, balloon catheter 100 could be moved further into cannula 410, and steerable element 132 could be articulated again. In various other embodiments, balloon catheter 100 could be moved further inward or outward relative to cannula 410 concurrently with the articulation of steerable element 132.

One balloon 120 is positioned as desired by steerable element 132, balloon 120 can be inflated as shown in FIG. 4C. This inflation can be performed by injecting an inflation fluid P (e.g., saline solution or contrast solution, among others) through connector 150. Then, when balloon 120 is deflated (inflation fluid P removed) as shown in FIG. 4D, a well-defined cavity 425 remains within cancellous bone 400-C.

Balloon catheter 100 can then be removed from vertebral body 400 by straightening steerable element 132 using actuator 140, or by simply allowing balloon 120 and steerable element 132 to be straightened as they are pulled through cannula 410, or by a combination of both.

Then, as shown in FIG. 4E, cavity 450 is filled with bone filler material 460 (e.g., PMMA) delivered by a nozzle 450 inserted through cannula 410. Bone filler material 460 can be expressed from nozzle 450 by any type of material delivery system, such as a syringe, plunger, and/or a hydraulic system among others. Note that while a nozzle having a side port is depicted for exemplary purposes, in various other embodiments, any type of delivery nozzle can be used (e.g., a open-ended nozzle or a multi-port nozzle, among others).

Once the filling operation is complete, delivery nozzle 450 and cannula 410 are removed from vertebra 400 (and the patient's body) as shown in FIG. 4F. Upon hardening, bone filler material 460 provides structural support for vertebra 400, thereby substantially restoring the structural integrity of the bone and the proper musculoskeletal alignment of the spine. Note that steerable element 132 of balloon catheter 100 allows bone filler material 460 to be delivered to a structurally advantageous location (e.g., towards the medial region of vertebral body 400) using a unilateral approach. This can beneficially reduce patient trauma compared to a typical bilateral kyphoplasty procedure while still providing the desired outcome.

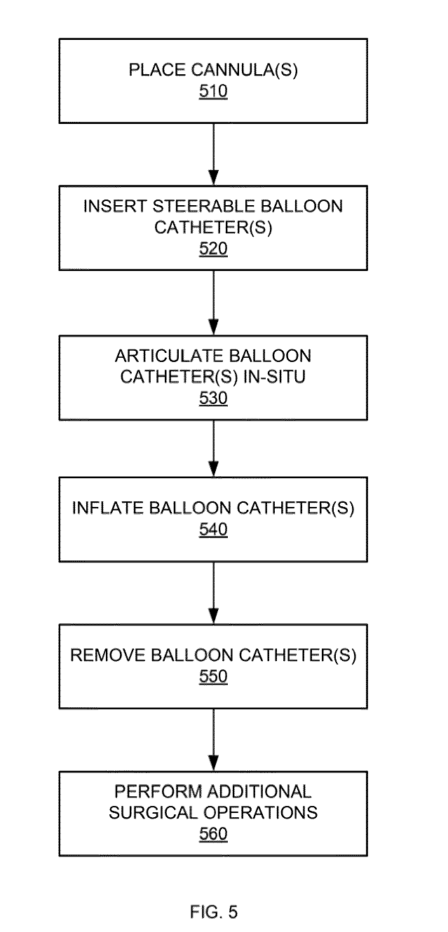

FIG. 5 shows a flow diagram of a process for performing a surgical procedure such as kyphoplasty using a balloon catheter incorporating an actively steerable element (as described with respect to FIGS. 1A-1B). In a PLACE CANNULA(S) step 510, one or more cannulas is positioned within a patient to provide a path to a target surgical location (e.g., as described with respect to FIG. 4A).

Then, in an INSERT STEERABLE BALLON CATHETER(S) step 520, one or more balloon catheters with an actively steerable element (e.g., as described with respect to FIGS. 1A-1B) is placed within the patient through the cannula(s) (e.g., as described with respect to FIG. 4A).

Next, in an ARTICULATE BALLOON CATHETER(S) IN-SITU step 530, the steerable element in each steerable balloon catheter can articulated to reposition the balloon catheter balloon (e.g., as described with respect to FIG. 4B). The balloon catheter(s) is (are) then inflated (e.g., as described with respect to FIG. 4C) in an INFLATE BALLOON CATHETER(S) step 540, with the steerable element at least partially controlling the inflation profile of the balloon. Note that in various embodiments, steps 530 and 540 can be performed any number of times, and in various orders, including simultaneously.

Then, in REMOVE BALLOON CATHETER(S) step 550, the balloon(s) are deflated and removed from the patient (e.g., as described with respect to FIG. 4D). Note that in some embodiments, steerable element can be articulated during this operation to simplify the removal process.

Finally, in a PERFORM ADDITIONAL SURGICAL OPERATIONS step 560, operations not involving the balloon catheter(s) can be performed to complete the procedure. For example, after removal of the balloon catheter from a bone, filler material can be delivered to the cavity formed by the balloon catheter (e.g., as described with respect to FIGS. 4E-4F).

Note that although the use of a balloon catheter incorporating an actively steerable steering element is described herein with respect to a kyphoplasty procedure for exemplary purposes, in various other embodiments, the steerable balloon catheter can be used in any other procedure that would benefit from such articulating capabilities.

For example, in some embodiments, a balloon catheter could be used to treat a long bone fracture. The steerable element could then allow the balloon to be optimally aligned in the long bone regardless of the particular access path used to initially place the balloon within the bone.

In various other embodiments, a steerable balloon catheter could be used to assess the open space within a vertebral disc. To treat back pain, a spinal fusion procedure is sometimes performed in which adjacent vertebrae are fused together. As part of the procedure, a portion of the intermediate disc nucleus material is removed for placement of an implant to assist the fusion. A balloon catheter with steerable element could be used immediately after the nucleus removal operation to determine the size and/or shape of the resulting nuclear space, with the steerability of the balloon catheter enabling optimized positioning for this diagnostic operation. Various other procedures that could benefit from an actively steerable balloon catheter will be readily apparent.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

1. A balloon catheter comprising:
an elongate shaft;
an inflatable element coupled to a distal end of the elongate shaft; and
an actively steerable element coupled to the inflatable structure; and
a controller at a proximal end of the elongate shaft for articulating the actively steerable element.

2. The balloon catheter of claim 1, further comprising a control shaft coupling the controller to the actively steerable element,
wherein the control shaft is positioned at least partially within the elongate shaft.

3. The balloon catheter of claim 2, wherein the actively steerable element extends into an interior of the inflatable structure.

4. The balloon catheter of claim 3, wherein a distal end of the actively steerable element is coupled to a distal end of the inflatable structure.

5. The balloon catheter of claim 1, further comprising:
a control shaft coupling the controller to the actively steerable element; and
an inner catheter positioned at least partially within the elongate shaft, and
wherein the control shaft is positioned at least partially within the inner catheter.

6. The balloon catheter of claim 5, wherein a distal end of the inner catheter is coupled to a distal end of the inflatable structure, and
wherein the actively steerable element is positioned at least partially within the inner catheter.

7. The balloon catheter of claim 1, further comprising a control shaft coupling the controller to the actively steerable element,
wherein the control shaft is positioned outside the elongate shaft, and
wherein the actively steerable element is coupled to an exterior surface of the inflatable structure.

8. The balloon catheter of claim 1, wherein the controller comprises a rotatable element for articulating the steerable element.

9. A kit comprising:
a cannula;
a balloon catheter sized to fit within the cannula, the balloon catheter comprising:
an elongate shaft;
an inflatable element coupled to a distal end of the elongate shaft; and
an actively steerable element coupled to the inflatable structure; and
a controller at a proximal end of the elongate shaft for articulating the actively steerable element.

10. The kit of claim 9, wherein the balloon catheter further comprises a control shaft coupling the controller to the actively steerable element,
wherein the control shaft is positioned at least partially within the elongate shaft.

11. The kit of claim 10, wherein the actively steerable element extends into an interior of the inflatable structure.

12. The kit of claim 11, wherein a distal end of the actively steerable element is coupled to a distal end of the inflatable structure.

13. The kit of claim 10, further comprising an inner catheter positioned at least partially within the elongate shaft, wherein the control shaft is positioned at least partially within the inner catheter.

14. The kit of claim 13, wherein a distal end of the inner catheter is coupled to a distal end of the inflatable structure, and
wherein the actively steerable element is positioned at least partially within the inner catheter.

15. A method of performing a surgical procedure, the method comprising:
placing a balloon catheter comprising an inflatable structure, an actively steerable element, and a controller at a target surgical location;
articulating the actively steerable element using the controller to change a configuration of the inflatable element; and
inflating the inflatable element.

16. The method of claim 15, wherein the balloon catheter further comprises:
an elongate element coupled to the inflatable structure; and
a controller shaft coupling the controller to the steerable element,
wherein the controller shaft is positioned at least partially within the elongate element, and
wherein the actively steerable element extends at least partially into the inflatable structure.

17. The method of claim 15, wherein the target surgical location comprises a region of cancellous bone within a fractured vertebral body,
wherein inflating the inflatable element creates a cavity in the region of cancellous bone, and
wherein the method further comprises delivering a quantity of bone filler material to the cavity.

18. The method of claim 17, wherein articulating the actively steerable element shears a portion of the region of cancellous bone.

19. The method of claim 17, wherein placing the balloon catheter comprises creating a percutaneous path to the target location using a cannula and inserting the balloon catheter into the cannula, the method further comprising:
deflating the inflatable element;
re-articulating the actively steerable element using the controller to place the actively steerable element into a substantially straight configuration; and
withdrawing the balloon catheter from the cannula.

20. The method of claim 15, wherein the target location comprises an interior region of a spinal disc,
wherein inflating the inflatable element comprises delivering a quantity of inflation fluid to the inflatable element, and
wherein the method further comprises:
assessing an open space within the spinal disc by at least one of visualizing the quantity of inflation fluid in the inflatable element and measuring the quantity of inflation fluid in the inflatable element.
